(19) United States
(12) Patent Application Publication
(10) Pub. No.: US 2009/0062599 A1
(43) Pub. Date: Mar. 5, 2009

(54) Transurethral cysto-urethropexy

(76) Inventor: John Brizzolara, Little Rock, AR (US)

Correspondence Address: Kyla D. Aycock;SPEED LAW FIRM, Suite 1200, 111 Center Street, Little Rock, AR, 72201, US

(21) Appl. No.: 12/079,540

(22) Filed: Mar. 27, 2008

Related U.S. Application Data
(60) Provisional application No. 60/966,336, filed on Aug. 27, 2007.

(51) Int. Cl. A61F 2/04; A61B 1/307

(52) U.S. Cl. 600/30; 600/135

(57) ABSTRACT

Embodiments include a method and apparatus for treating stress urinary incontinence transurethrally. In one embodiment, the method includes entering the urethra of a patient through a urethra opening from the patient's body and connecting a bladder to a fascia using an apparatus and one or more connecting members, which may optionally be connected to a remote viewing apparatus. Embodiments of the apparatus may include a curved instrument capable of placing one or more connecting members through a bladder wall and through the fascia. In some embodiments, minimal or no incisions are made in a body of the patient.

### CROSS-REFERENCE TO RELATED APPLICATIONS

This application claims benefit of U.S. Provisional Patent Application Ser. No. 60/966,336, filed Aug. 27, 2007 and entitled “Transurethral Cysto-Urethropexy,” which is herein incorporated by reference in its entirety.

### BACKGROUND OF THE INVENTION

1. Field of the Invention

Embodiments generally relate to treatment for urinary incontinence. More specifically, embodiments relate to treatment for stress urinary incontinence.

2. Description of the Related Art

Stress urinary incontinence is a common problem which may be caused by increased pressure in the abdominal cavity. Specifically, incontinence may be caused by the urinary sphincter, the muscle that encircles the urethra, losing the ability to stop urine exiting the urethra.

Various surgical procedures have been developed for treating urinary incontinence. Previously, the available surgical treatments have been the following: (1) transabdominal cystopexy (Marshall Marshete Krantz (“MMK”), Burch urethropexy, Lapedes anterior cystopexy), all requiring an abdominal incision; (2) laparoscopic Burch urethropexy, requiring transabdominal placement of laparoscopic instruments, and (3) sub-urethral slings.

The first two options, the transabdominal cystopexy procedures and the laparoscopic Burch urethropexy procedure, are disadvantageous because they require incisions into the abdominal or retroperitoneal cavity, increasing morbidity chances due to the incisions and risking injury to the bowel or vascular structures due to the incisions. Additionally, these procedures require a hospital stay of usually one to two nights due to the relative severity of the procedures.

The third option, the sling procedure (such as the pubo-vaginal sling or trans-obturator sling), usually involves placing tissue or graft from a patient or animal like a hammock on the urethra. One type of sling procedure requires a smaller incision and a tiny incision on the vagina underneath the urethra to place the sling, while another type of sling procedure requires at least three total incisions to install the sling. The sling procedure requires dissection underneath the urethra, so that when the sling procedure is used in women of child-bearing age, it may preclude the use of subsequent sub-urethral slings for recurrent stress urinary incontinence or may negatively impact the success of a second sling procedure.

Therefore, there is a need for a less invasive and less dangerous treatment for stress urinary incontinence. There is a further need for a procedure for treating stress urinary incontinence which does not prevent effective treatment for recurring stress incontinence. There is yet a further need for a stress urinary incontinence treatment procedure which does not require incisions into the abdominal or retroperitoneal cavity.

### SUMMARY OF THE INVENTION

Embodiments generally include a method for treating urinary incontinence in a patient, comprising entering into an interior of a urethra of the patient's body with an apparatus through a urethra opening from the body; elongating the urethra towards a fascia operatively connected to a pubic bone using the apparatus; and connecting a bladder of the patient to the fascia by using the apparatus to install one or more connecting members. Further embodiments generally include an instrument for treating urinary incontinence transurethrally in a patient's body, comprising an elongated remote viewing apparatus for obtaining images within the body; a curved piercing member operatively connected at or near an end of the elongated viewing apparatus; and a display capable of receiving and displaying images from within the body obtained by the remote viewing apparatus.

### BRIEF DESCRIPTION OF THE DRAWINGS

So that the manner in which the above-recited features of embodiments of the present invention can be understood in detail, a more particular description of the invention, briefly summarized above, may be had by reference to embodiments, some of which are illustrated in the appended drawings. It is to be noted, however, that the appended drawings illustrate only typical embodiments of this invention and are therefore not to be considered limiting of its scope, for the invention may admit to other equally effective embodiments.

FIG. 1 is a longitudinal cross-sectional view through a portion of the pelvic region of a female patient, showing a remote viewing device such as a cystoscope and an apparatus inserted through the interior of the urethra.

FIG. 2 is a longitudinal cross-sectional view of the apparatus of FIG. 1 positioned within the urethra and in the process of stretching and elongating the urethra.

FIG. 3 is a longitudinal cross-sectional view of the apparatus of FIG. 1 connecting the bladder and bladder neck to the fascia transurethrally and stabilizing the urethra.

FIG. 4 is a longitudinal cross-sectional view of the pelvic region of FIG. 1 showing the bladder supported by a connecting member after the apparatus is removed from the body.

FIG. 5 is a longitudinal cross-sectional view of through a portion of the pelvic region of a female patient, illustrating an alternate embodiment.

### DETAILED DESCRIPTION

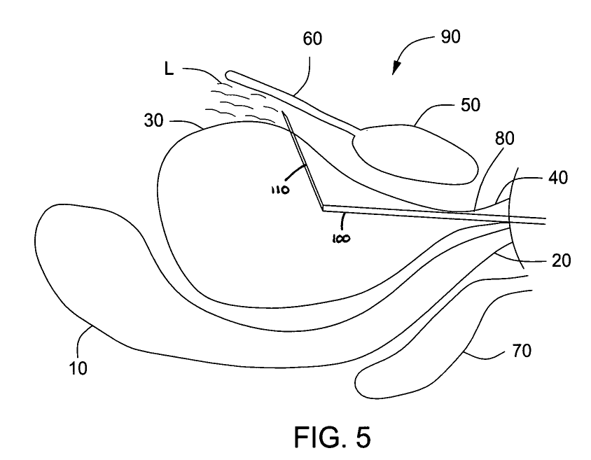

Embodiments include a cystoscopically-assisted transurethral cysto-urethropexy which may be incisionless. Additionally, embodiments include a transurethral urethropexy and/or cystopexy (transurethral cysto-urethropexy) procedure using one or more connecting members (for example absorbable staples, threads, stitches, and/or sutures) to treat stress urinary incontinence in women primarily of child-bearing age. The procedure may be performed under direct cystoscopic vision with or without the aide of fluoroscopy (a fluoroscope is a type of imaging technique, which may include x-ray imaging, known to those skilled in the art) or any other imaging or x-ray method or device known to those skilled in the art. The one or more connecting members (for example absorbable staples, threads, stitches, and/or sutures) may be placed through the anterior bladder “neck” and through the periosteum of the posterior surface of the pubic symphysis, the objective being to elevate, elongate, and fix the urethra. One or more connecting members (for example absorbable staples, threads, stitches, and/or sutures) may be placed through the anterior bladder wall into the rectus muscle (fascia) to provide further stabilization and fixation of the bladder and urethra. Prior to placement of connecting members, as shown in an alternate embodiment shown in FIG. 5, a solution may be injected into the space anterior to the bladder wall and posterior to the rectus fascia, the objective being to facilitate adherence of the anterior bladder wall to the rectus fascia.

The procedure of embodiments has the following advantages over the previously-performed stress urinary incontinence treatment procedures: the procedure is minimally invasive, performable on an outpatient basis, and does not require an abdominal incision or placement of laparoscopic instruments, thereby avoiding the morbidity associated with these incisions and further avoiding the possible injury to bowel or vascular structures. The use of the sub-urethral sling is advantageously reduced to use in women after they are no longer of child-bearing age. Also advantageously, the procedure of the present invention incorporates the long-term 85% success rate of the more invasive cysto-urethropexies into this minimally invasive procedure.

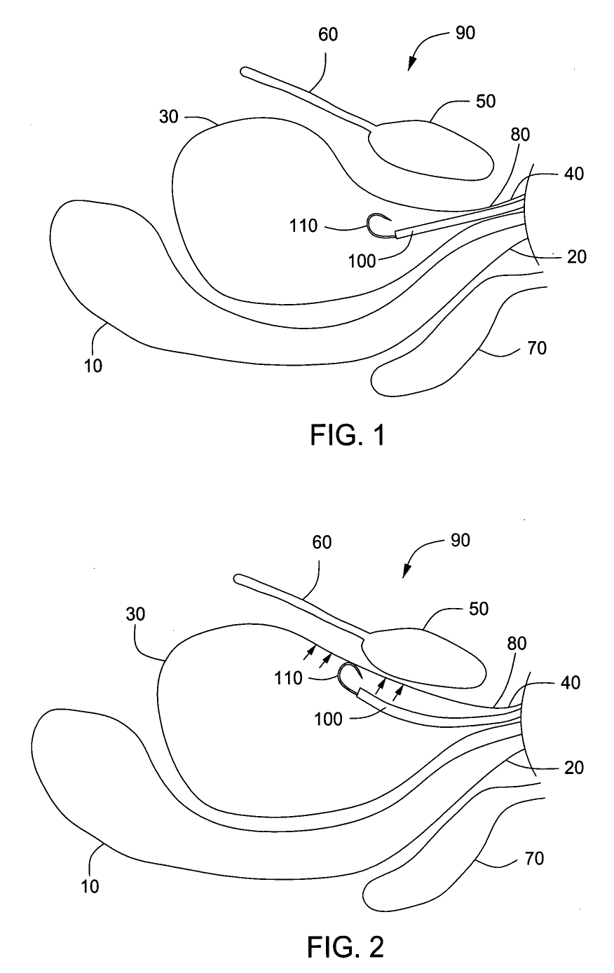

FIG. 1 illustrates a portion of a pelvic region 90 of a female patient. The pelvic region 90 includes the bladder 30 which meets the urethra 40 at a bladder neck 80, also called an urethrovesical junction. A uterus 10 which leads to a vagina 20 is disposed generally adjacent to the bladder 30, while the vagina 20 is disposed generally adjacent to the urethra 40. A rectum 70 is disposed below the vagina 20. Located above the urethra 40 is the pubic bone 50, also called symphysis pubis. Attached to the pubic bone 50 is fascia 60, a type of tissue.

Urinary stress incontinence is caused when a patient's bladder neck 80 loses support, stabilization, and/or slight compression to eliminate unwanted loss of urine, for example due to coughing, laughing, or other events which increases intra-abdominal pressure on the bladder 30. The urinary sphincter, the muscle that encircles the urethra, usually stops urine, but in urinary stress incontinence the muscle may not possess the ability to stop the urine from exiting the urethra.

FIG. 1 also shows a remote viewing apparatus 100 such as a cystoscope having an apparatus or instrument 110 of embodiments operatively attached thereto, where the instrument 110 and at least a portion of the cystoscope 100 are disposed within the bladder 30. The cystoscope 100 is capable of taking/gathering images from within a patient's body and relaying these images to a computer for display on a monitor or other apparatus capable of displaying an image from a computer readable medium, allowing the doctor performing the procedure to know where the instrument 110 is located within the patient's body and where to navigate the instrument 110 within the body to accomplish the procedure. Instead of the cystoscope, direct vision may be used. One or more x-ray devices may optionally be included for use with the remote viewing apparatus 100, for example one or more fluoroscopes for performing fluoroscopy.

In one embodiment, the apparatus 110 is preferably at least partially curved to allow it to travel through a wall of the bladder 30, through the fascia 60, and curve around back through the wall of the bladder 30 without having to remove the instrument 110 from within the bladder 30, as described below. In the embodiment shown in FIGS. 1-4, the apparatus 110 includes a piercing or sharp instrument such as a needle which is generally u-shaped to allow threading of one or more sutures, stitches, or other thread-like objects known to those skilled in the art through the bladder 30 wall and fascia 60, as described below.

In operation, the transurethral cysto-urethropexy may be performed as follows. The instrument 110 may be operatively connected to the viewing apparatus 100 to permit the instrument 110 and viewing apparatus 100 to travel as a unit into the urethra 40. The instrument 110 and cystoscope 100 are then inserted into the interior of the urethra 40 (transurethrally) through the opening of the urethra 40 from the body and into the bladder 30, as shown in FIG. 1.

During the procedure, the person performing the procedure may view the positioning and location of the instrument 110 using the display monitor when the cystoscope 100 (or other viewing device) gathers the positioning/location information and relays that information to the computer or computer readable medium, which information is displayed on the display monitor/member or direct vision. Any type of cystoscope, x-ray, computer, and/or accessories to the cystoscope, x-ray, and/or computer which are known to those skilled in the art may be utilized to provide real-time location and positioning information gathered from the inside of the body and display this information outside the body.

The person performing the surgery moves the instrument 110 into position via the cystoscope 100 portion that remains outside the body until the instrument 110 is adjacent the portion of the urethra 40 or bladder 30 which is to be tacked or operatively connected to the fascia 60 and/or pubic symphysis, preferably via the fascia 60. As shown in FIG. 2, the instrument 110 is utilized, for example by being pressed against the wall of the bladder 30 and/or urethra 40, to stretch and elongate the urethra 40.

Next, the anterior bladder wall 30 and/or the urethra 40 is secured to the rectus fascia 60 and/or to the pubic symphysis 50 (pubic bone) by one or more connecting members such as sutures, threads, stitches, and/or staples. In one embodiment, a needle or other piercing member, such as that associated with or connected to the instrument 110, may be used to thread one or more sutures, threads, or stitches from the inside of the bladder 30 or urethra 40 through the wall of the bladder 30 or urethra 40, into and through the fascia 60 and/or pubic symphysis, out from the fascia 60 and/or pubic symphysis, and from the outside of the bladder 30 or urethra 40 through the wall of the bladder 30 or urethra 40 and back inside the bladder 30 or urethra 40. A curved instrument 110 allows a needle to travel in, out, and over the wall of the bladder 30 and/or urethra 40 to in and out the fascia to facilitate the passing of the suture(s) through these structures.

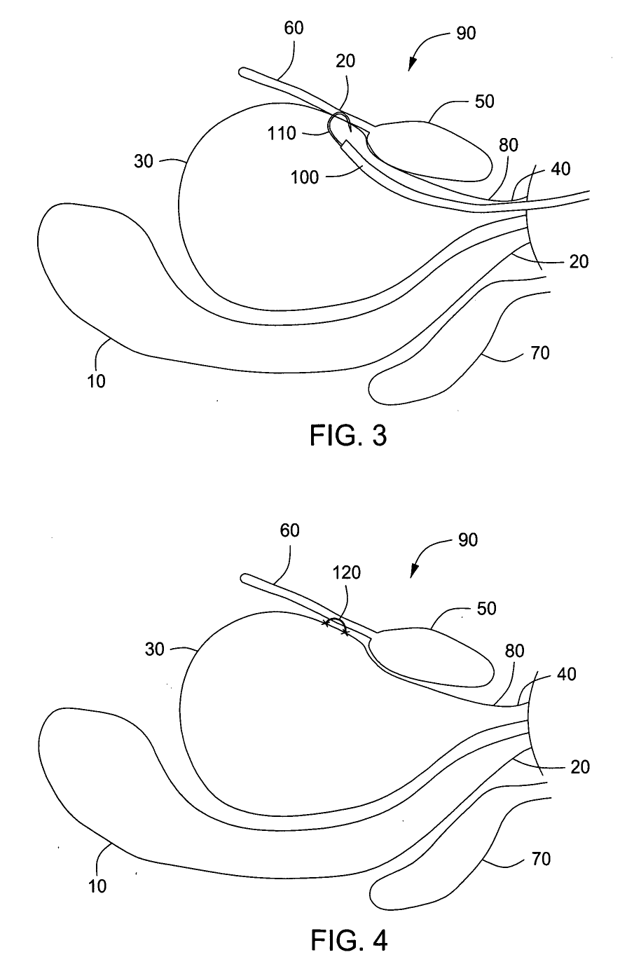

The one or more connecting members 120 (e.g., thread, sutures, staples, and/or stitches) are placed from the inside through the wall of the bladder 30 and/or urethra 40 and out the wall, over the wall through the fascia 60 and/or pubic symphysis, and back through the wall of the bladder 30 and/or urethra 40 to the inside of the bladder 30 and/or urethra 40, as illustrated in FIG. 3. The one or more connecting members 120 are secured into position within the bladder 30/urethra 40, for example by tying the thread, sutures, and/or stitches within the inside of the bladder 30 and/or urethra 40 or by any other means of securing thread, sutures, or stitches into position known to those skilled in the art. The instrument 110 and cystoscope 100 are then removed from the inside of the bladder 30 and urethra 40, leaving the connecting member 120 within the body.

The one or more connecting members may optionally be one or more absorbable threads, sutures, and/or stitches. The instrument 110 may for example possess the same angle of curvature as a Lowsley retractor, typically used for prostate surgery or Van Buren sounds which may be utilized to dilate urethras.

In an alternate embodiment, the instrument 110 may include or be substituted with a face plate or other guiding member, which may be absorbable, through which one or more tacking devices, for example one or more button pushers, may place one or more staples, threads, sutures, and/or stitches through the face plate into the fascia 60 (and/or pubic symphysis) and through the wall of the urethra 40 and/or bladder 30. This face plate and button pusher may be utilized in lieu of or in conjunction with the instrument 110 shown in FIGS. 1-4. The tacking device may optionally be utilized to fire the face plate into position as well as push the thread through the face plate.

Ultimately, the entire urethra area is elongated and stabilized when the one or more connecting members 120 (which may be staples, threads, sutures, stitches, or any other connecting members known to those skilled in the art) are placed through the bladder and/or urethra, the fascia 60, and/or pubic symphysis. Placing the one or more connecting members 120 through the fascia 60 according to embodiments disclosed herein lowers the risk of ostetitis pubis, which is infection of the pubic bone which often results when connecting members 120 are placed through the pubic bone.

An alternate embodiment may include injecting a liquid substance L into the space between the anterior bladder wall and the rectus fascia, as shown in FIG. 5. This space is composed of fatty tissue, and the liquid would disturb this space. By doing so, after the connecting member or members have been placed, the result would be adherence (scarring) of the anterior bladder wall to the rectus fascia. The instrument 110 may be configured in many different ways, as depicted in FIG. 5.

A further alternate embodiment includes disturbing the space between the anterior bladder wall and the rectus fascia by delivering heat to the area by radio frequency, laser, electricity, and/or any other method or apparatus for delivering heat to the space, rather than or in addition to injecting the liquid substance into the space.

Embodiments described herein advantageously provide an incisionless procedure for treating urinary incontinence, particularly for treating stress urinary incontinence. This incisionless procedure is less invasive to the patient than prior procedures, thereby decreasing the danger of treatment for stress urinary incontinence and decreasing overnight stays at medical facilities. Furthermore, embodiments advantageously do not prevent effective future treatment for recurring stress incontinence.

While the foregoing is directed to embodiments of the present invention, other and further embodiments of the invention may be devised without departing from the basic scope thereof, and the scope thereof is determined by the claims that follow.

1. A method for treating urinary incontinence in a patient, comprising:
entering into an interior of a urethra of the patient's body with an apparatus through a urethra opening from the body;
elongating the urethra towards a fascia operatively connected to a pubic bone using the apparatus; and
connecting a bladder of the patient to the fascia by using the apparatus to install one or more connecting members.

2. The method of claim 1, wherein connecting the bladder to the fascia comprises:
placing the one or more connecting members from an inside of the bladder through a wall of the bladder to an outside of the bladder, over the outside of the wall of the bladder, through the fascia, and from an outside of the bladder through the wall of the bladder.

3. The method of claim 2, wherein the apparatus is used for the placing the one or more connecting members.

4. The method of claim 3, wherein the apparatus is at least partially curved at its end to permit the placing of the one or more connecting members with the apparatus using the at least partially curved end.

5. The method of claim 4, wherein the apparatus is a Lowsley retractor.

6. The method of claim 4, wherein the at least partially curved end of the apparatus comprises a partially curved needle which carries the one or more connecting members through and over the wall of the bladder and through the fascia.

7. The method of claim 6, wherein the needle is generally unshaped.

8. The method of claim 1, wherein the method is accomplished without any incisions through a body of the patient.

9. The method of claim 1, wherein the one or more connecting members comprise one or more sutures, stitches, threads, staples, or a combination thereof.

10. The method of claim 9, wherein the one or more connecting members are absorbable.

11. The method of claim 1, wherein the apparatus is operatively connected to a remote vision mechanism which is operatively connected to a display, allowing remote visual guidance of the apparatus within the urethra.

12. The method of claim 11, wherein the remote vision mechanism is a cystoscope.

13. The method of claim 12, wherein one or more imaging devices are used in conjunction with the cystoscope.

14. The method of claim 1, wherein the connecting the bladder to the fascia using the apparatus to install one or more connecting members comprises using one or more face plates for guiding and allowing placing of one or more connecting members therethrough using the apparatus.

15. The method of claim 14, wherein the one or more face plates are absorbable.

16. The method of claim 1, wherein the apparatus uses the one or more connecting members to connect the bladder to the fascia without the aid of any other device.

17. An instrument for treating urinary incontinence transurethrally in a patient's body, comprising:
an elongated remote viewing apparatus for obtaining images within the body;
a curved piercing member operatively connected at or near an end of the elongated viewing apparatus; and
a display capable of receiving and displaying images from within the body obtained by the remote viewing apparatus.

18. The apparatus of claim 17, wherein the curved piercing member comprises a generally u-shaped needle.

19. The apparatus of claim 17, further comprising one or more connecting members operatively connected to the piercing member, the piercing member being capable of locating the one or more connecting members through a bladder wall and through a fascia or pubic bone of the patient's body.

20. The apparatus of claim 19, wherein the piercing member is capable of entering an inside of the urethra through a urethra body opening, guiding the one or more connecting members from inside a bladder to outside the bladder through the bladder wall and through the fascia or pubic bone, and guiding the one or more connecting members from the outside of the bladder through the bladder wall to the inside of the bladder.
